(19) United States
(12) Patent Application Publication
(10) Pub. No.: US 2021/0071208 A1
(43) Pub. Date: Mar. 11, 2021

(54) SELECTIVE PRODUCTION OF 1,3-PROPANEDIOL MONOACETATE

(71) Applicant: DSM IP ASSETS B.V., Heerlen (NL)

(72) Inventors: Henricus Martinus Maria Gerardus STRAATMAN, Geleen (NL); Martin SCHÜRMANN, Geleen (NL); Peter Hans RIEBEL, Kaiseraugst (CH)

(21) Appl. No.: 16/958,859

(22) PCT Filed: Dec. 14, 2018
(86) PCT No.: PCT/EP2018/084939

§ 371 Date: Jun. 29, 2020

(30) Foreign Application Priority Data
Dec. 31, 2017 (CH) 01628/17

(51) Int. Cl. C12P 7/18

(52) U.S. Cl. CPC C12P 7/18; C12Y 301/01003

(57) ABSTRACT

The present invention is related to a novel selective one-step enzymatic process for hydrolysis of 1,3-propanediol diacetate (PDDA) into 1,3-propanediol monoacetate (PDMA).

The present invention is related to a novel selective one-step enzymatic process for hydrolysis of 1,3-propanediol diacetate (PDDA) into 1,3-propanediol monoacetate (PDMA).

PDMA is an important intermediate in the production of 1,3-propanediol mononitrate (PDMN), a compound that has been reported to be highly efficient in reducing the formation of methane in ruminants. Ruminants, in particular cattle, are besides the fossil fuel industry the major contributors to the biogenic methane formation leading to global warming or climate change. It has been estimated that the prevention of methane formation from ruminants would almost stabilize atmospheric methane concentrations.

As disclosed in WO2012/084629, PDMN can be prepared by the reaction of 3-bromopropanol in acetonitrile with silver nitrate.

Such a process, however, is neither ecological acceptable nor feasible for industrial scale production.

Thus, it is an ongoing task to develop an efficient, economic, eco-friendly and safe industrial process for production of PDMN via mono-hydrolysis of PDDA, which avoids the use of e.g. halogenated precursors and/or raw materials and is amenable to scale-up. Furthermore, the amount of 1,3-propanediol (PD) which might be a possible by-product of such conversions should be reduced or its formation abolished.

Surprisingly, we now identified enzymes involved in the selective mono-hydrolysis of PDDA into PDMA which can be further processed into PDMN.

Particularly, the present invention is directed to the use of carboxylic ester hydrolases [EC 3.1.1] in a catalytic process for production of PDMA, said enzymes catalyzing the mono-hydrolysis of PDDA into PDMA, with high selectivity and productivity meaning a PDDA-conversion of at least about 75% and a PDMA yield of at least about 75%.

Thus, in one aspect the present invention is directed to a process for conversion of PDDA into PDMA, said conversion being a selective mono-hydrolysis of PDDA into PDMA being catalyzed by an enzyme having carboxylic ester hydrolase [EC 3.1.1] activity, such as e.g. enzymes with esterase or lipase activity.

For the purpose of the present invention, any enzyme [EC 3.1.1] can be used for the conversion of PDDA into PDMA as long as the putative enzyme is capable of selective 1-step mono-hydrolysis of PDDA. Particularly, the enzyme is selected from esterase or lipase, preferably lipases [EC 3.1.1.3], more preferably a Candida lipase, most preferably Candida antarctica lipase B (CalB). CalB is commercially available from various suppliers.

The terms “lipase or esterase”, “enzyme having lipase or esterase activity”, “PDDA hydrolyzing enzyme” or “PDDA mono-deacetylating enzyme” are used interchangeably herein. It refers to an enzyme having lipase/esterase activity which is involved in conversion, i.e. one-step mono deacetylation, of PDDA into PDMA as defined herein. The terms “CalB” and “Candida antarctica lipase B” are used interchangeably herein.

The terms “conversion”, “hydrolysis”, “deacetylation” in connection with enzymatic catalysis of the enzymes [EC 3.1.1] as described herein leading to production of PDMA from PDDA, are used interchangeably herein.

As used herein, the term “specific activity” or “activity” with regards to enzymes means its catalytic activity, i.e. its ability to catalyze formation of a product from a given substrate. The specific activity defines the amount of substrate consumed and/or product produced in a given time period and per defined amount of protein at a defined temperature. Typically, specific activity is expressed in μmol substrate consumed or product formed per min per mg of protein. Typically, μmol/min is abbreviated by U (=unit). Therefore, the unit definitions for specific activity of μmol/min/(mg of protein) or U/(mg of protein) are used interchangeably throughout this document. An enzyme is active, if it performs its catalytic activity in vivo, i.e. within the host cell as defined herein or within a system in the presence of a suitable substrate. The skilled person knows how to measure enzyme activity, in particular activity of esterases or lipases, such as in particular CalB activity, as defined herein. Analytical methods to evaluate the capability of a suitable enzymes as defined herein for PDMA production from conversion, i.e. mono-deacetylation, of PDDA are known in the art.

The enzymatic conversion of PDDA to PDMA, i.e. selective mono-deacetylation, as described herein can be performed with isolated enzymes, such as e.g. lipases or esterases, in particular lipases, such as e.g. CalB, as defined herein which might be expressed in a suitable host cell, either as endogenous or heterologous enzymes. The expressed enzyme (lipases or esterase) might be produced intracellularly or excreted to the fermentation broth. Isolation of suitable enzymes can be done by known methods, the respective enzymes might be further used in isolated form or in the form of a cell extract, powder or liquid formulations, i.e. in immobilized or non-immobilized form. Preferably, the enzymes, in particular lipases, such as e.g. CalB, that are used in a process for selective mono-deacetylation of PDDA to PDMA are used in liquid or immobilized form, such as either covalently bound or adsorbed, depending on the supplier. When using immobilized forms, the enzymes might be used several times with more or less the same performance (so-called recycling).

For isolation and purification of said enzymes, e.g. esterase or lipase as defined herein, the cells of the microorganism may be harvested after cultivation from the liquid culture broth by for instance centrifugation or filtration. The harvested cells may be washed for instance with water, physiological saline or a buffer solution having an appropriate pH. The washed cells may be suspended in an appropriate buffer solution and disrupted by means of for instance a homogenizer, sonicator, French press, or by treatment with lysozyme and the like to give a solution of disrupted cells. In case of excreted enzymes, the target enzyme might be isolated from the fermentation broth directly or the cell-free supernatant of a fermentation after for instance centrifugation or filtration. The suitable enzymes, such as e.g. CalB, may be isolated and purified from the cell-free extract or disrupted cells, differential solubilization using appropriate detergents, precipitation by salts or other suitable agents, dialysis, ion exchange chromatographies, hydroxyapatite chromatographies, hydrophobic chromatographies, size exclusion chromatographies, affinity chromatographies, or crystallization. When the suitable enzyme is produced as tagged polypeptide such as for instance a His-tag one, it may be purified with affinity resins such as for instance Nickel affinity resin. The purification of said enzymes may be monitored photometrically by using for instance model substrates such as para-nitro-acetate, para-nitro-decanoate or para-nitro-palmitate.

With regards to the present invention, it is understood that organisms, such as e.g. microorganisms, fungi, algae or plants also include synonyms or basonyms of such species having the same physiological properties, as defined by the International Code of Nomenclature of Prokaryotes or the International Code of Nomenclature for algae, fungi, and plants (Melbourne Code).

In one embodiment, the enzyme to be used for selective mono-deacetylation of PDDA into PDMA is a lipase, in particular a Candida lipase, such as e.g. Candida antarctica lipase B, which is capable of mono-hydrolysing PDDA into PDMA with a conversion of at least about 75%, such as e.g. at least about 80, 85, 90, 92, 95, 97, 98, 99 or even 100% and a PDMA yield of at least about 75%, such as e.g. at least about 80, 85, 90, 92, 95, 97, 98, 99 or even 100%, depending on the amount of substrate (i.e. PDDA), enzyme concentration/enzyme form or suitable reaction conditions. The enzyme might be used as liquid or immobilized formulations, such as commercially available from e.g., Novozymes, Chiral Vision, Fermenta or CLEA Technology. Preferably, the enzyme is reused or recycled for several hydrolysis-reactions, such as e.g. at least 4, 6, 8, 9, 10 or more reactions, in particular when using the immobilized forms.

In one embodiment of the present invention, the conversion of PDDA into PDMA via enzymatic mono-deacetylation using an esterase or lipase [EC 3.1.1], such as e.g. a lipase [EC 3.1.1.3], including but not limited to Candida lipase, such as e.g. lipase B from Candida antarctica, which is used in the form or a liquid enzyme of immobilized, either adsorbed or covalently bound, as defined herein, is performed in a suitable medium, such as e.g. a medium comprising PDDA in an amount of less than about 40 wt %, such as less than about 35, 25, 20, 18 wt %, such as e.g. in the range of about 12 to about 18 wt %, in particular about 12, 13, 14, 15, 16, 17 wt % PDDA, at a suitable temperature, such as e.g. at least about 25° C. but no more than 38° C., such as e.g. about 26, 27, 28, 30, 32, 34, 36, 37, 38° C., in particular between about 28 and 37° C., at a suitable pH, such as e.g. in a range about 7 and 8, such as e.g. 7.0, 7.2, 7.5, 7.7, 7.8, 8.0° C., for a suitable time, such as e.g. at least about 1 h, 2 h, 3 h, 4 h or more, such as e.g. 6 h, 8 h, 10 h or up to 20 h and more, depending on the activity of the enzyme, in the presence of suitable buffers or titrants such as co NaHCO₃ or NaOH, such as e.g. 5M NaOH, as titrant, i.e. “suitable conversion conditions” as used herein.

Thus, according to the present invention the substrate PDDA is contacted with an esterase or lipase, particularly a lipase, such as from Candida antarctica e.g. CalB, as defined herein, leading to mono-deacetylation into PDMA under suitable conversion conditions as defined above. Preferably, the amount of substrate is in the range of 17 wt % or less, such as e.g. about 12, 14, 15, 16 wt % PDDA, with an optimal range of about 14 to 15 wt % PDDA which is contacted preferably with at least about 0.8, 0.9, 1.0, 1.2, 1.5, 1.7, 1.8, 2.0, 2.2, 2.5 mg or even 3, 6, 9 mg, such as e.g. CalB, per ml reaction, as defined herein. When using immobilized enzyme forms, the enzyme is preferably used several times, i.e. recycled, with at least 5, 6, 7, 8, 9, 10 or more recycling reactions.

With such process as described above, a conversion rate of at least about 75%, such as e.g. at least about 80, 85, 90, 92, 95, 97, 98, 99 or even 100%, resulting to yields of at least about 86% or even more after a reaction for at least about 1 h or even up to 8 h and more under suitable conversion conditions as defined herein is achieved.

In particular, the present invention features the following embodiments:

(1) A process for production of 1,3-propanediol monoacetate (PDMA) comprising mono-deacetylation of 1,3-propanediol diacetate (PDDA) catalyzed by carboxylic ester hydrolases [EC 3.1.1], preferably esterases or lipases, more preferably lipases [EC3.1.1.3], in particular Candida lipase, preferably Candida antarctica lipase B (CalB).

(2) Use of these carboxylic ester hydrolases [EC 3.1.1], preferably esterases or lipases, more preferably lipases [EC3.1.1.3], for mono-deacetylation of PDDA into PDMA.

(3) Process or use as above and as defined herein, wherein the lipase is used in liquid or immobilized form, in particular wherein the immobilized enzyme is adsorbed or covalently bound, particularly wherein the enzyme is re-used for at least 5 times.

(4) Process or use as above and as defined herein, wherein PDDA is converted into PDMA at a rate of at least about 75%.

(5) Process or use according as above and as defined herein, wherein the mono-deacetylation reaction is in the presence of NaHCO₃.

(6) Process or use as above and as defined herein, wherein the mono-denitrification reaction is conducted at pH in the range of 7 to 8.

(7) Process or use as above and as defined herein, wherein the mono-deacetylation reaction is conducted at 28° C.

(8) Process or use as above and as defined herein, wherein the amount of PDDA is less than 40 wt %.

The following examples are illustrative only and are not intended to limit the scope of the invention in any way. The contents of all references, patent applications, patents, and published patent applications, cited throughout this application are hereby incorporated by reference.

### EXAMPLES

### Example 1: Materials and General Methodology

All chemicals used were of analytical grade. For enzyme reactions, cell-free extracts (cfe), powder or liquid formulations were used. Protein content and expression level were determined by SDS-PAGE. The enzymes used were either in the form of cfe, powder or liquid formulation, depending on the supplier. Liquid forms of CalB were purchased from Novozymes, immobilized forms purchased from Novozymes or Chiral Vision.

Formation of PDMA and PDDA was measured via GC analysis according to the following protocol (Table 1), wherein samples of 250 μl were accurately mixed with 750 μl of a THF-water mixture (75% THF:25% H₂O) and analyzed.

TABLE 1
Settings of GC analysis for PDMA and PDDA. Internal standard:
2-methyl-1-butanol; solvent: acetonitrile; internal standard
solution concentration: 2 mg/ml acetonitrile; sample solution:
ca. 100-200 mg in 5 ml internal standard solution.

| Column | Cp sil 8cb 25 m × 0.25 μm df = 1.2 μm |
|---|---|
| Injection | split |
| Flow | 1.2 mL/min |
| Split ratio | 20 |
| Total flow | 23.7 ml/min |
| Injector temperature | 250° C. |
| Detector temperature | 300° C. |
| Initial temperature | 100° C. |
| Initial time | 1 min |
| Column | Cp sil 8cb 25 m × 0.25 μm df = 1.2 μm |
| Rate 1 | 5° C. |
| Temperature 2 | 175° C. |
| Rate 2 | 25° C./min |
| Final temperature | 250° C. |
| Final time | 0 min |

During the reactions time samples were taken. The conversions, in mol %, were calculated based on recovery because of the sometimes not homogeneous sample taking and deviations in the mass balance. From reaction perspective, this can be done because PDMA and PD are the only products that can be formed during the reaction.

Due to the formation of acetic acid during PDDA hydrolysis, the experiments were carried out in pH stat equipment with continuous monitoring and pH adjustment with e.g. NaOH.

### Example 2: Testing of PDDA Hydrolyzing Enzyme CalB (Liquid Form)

In order to determine the best reaction conditions, a liquid formulation of CalB was tested with NaOH as titrant (Table 2A). Testing was performed in 10 ml reactions at pH 7.5 using various amounts of PDDA as substrate. The result is shown in Table 2B.

TABLE 2A
Protocol applied for reactions with
CalB in 10 ml reactions at 28° C.

| Compound | Amount | | | |
|---|---|---|---|---|
| PDDA | 14.8 | wt % | 36.1 | wt % |
| Potassium phosphate | 8.5 | ml | 5 | ml |
| (KPᵢ) buffer | | | | |
| 50 mM pH 7.5 | | | | |
| PDDA (91%) | 1.65 | g | 3.3 | g |

| | |
|---|---|
| CalB liquid | 60 mg |
| NaOH | 5M |

TABLE 2B
Conversion of PDDA to PDMA using CalB under
the conditions described in Table 2A.

PDDA [wt %]

| | 14.8 | 36.1 |
|---|---|---|
| Initial activity (U/mg; after 0.5 h) | 3.1 | 2.8 |
| Hydrolysis ration PDDA/PDMA | 17 | |
| Reaction time (h) | 2 | 17 |
| Composition mixture (mol %) | | |
| PDMA | 86 | 64 |
| PD | 9 | 30 |
| PDDA | 5 | 6 |
| Concentration PDMA end reaction (wt %) | 7 | 11 |
| Productivity (g PDMA/L*h) | 37 | 17 |
| Enzyme usage (kg/kg PDMA) | 0.06 | |

The results show that the reaction with CalB and 14.8 wt % PDDA proceeded very fast. In 2 h, 95% conversion was reached with 60 mg of enzyme. The hydrolysis ratio PDDA/PDMA was very high leading to 86% PDMA yield. A drastic increase in PDDA concentration to 36 wt % lead to deactivation of the enzyme, much longer reaction time and decrease in selectivity.

### Example 3: Reaction Optimization for CalB (Liquid Form)

Based on the results of Example 2, further experiments were set-up with 14.8 wt % PDDA as substrate but with 30, 60 or 90 mg of CalB liquid (for protocol, see Table 2A) leading to a final enzyme concentration of 3, 6 or 9 mg/ml CalB liquid. The results are shown in Table 3.

TABLE 3
Conversion of PDDA to PDMA using different amounts of CalB liquid.
Composition reaction mixture (mol %)

| Reaction | 30 mg CalB liquid | | | 60 mg CalB liquid | | | 90 mg CalB liquid | | |
|---|---|---|---|---|---|---|---|---|---|
| time (h) | PD | PDMA | PDDA | PD | PDMA | PDDA | PD | PDMA | PDDA |
| 0 | 0 | 0 | 100 | 0 | 0 | 100 | 0 | 0 | 100 |
| 0.16 | 0.3 | 17 | 82.6 | 0.7 | 34.4 | 64.8 | 1.0 | 39 | 60 |
| 0.5 | 0.8 | 35.7 | 63.5 | 1.9 | 60 | 38.1 | 2.6 | 63.9 | 33.5 |
| 1 | 1.5 | 50.9 | 47.6 | 4.1 | 77.8 | 18 | 4.7 | 79.4 | 16 |
| 2 | 3.2 | 71.5 | 25.3 | 8.7 | 86 | 5.3 | 8.9 | 85.5 | 5.6 |
| 3 | | | | 15.9 | 81 | 3.1 | | | |
| 3.7 | 5.9 | 83.5 | 10.6 | | | | 15.3 | 81.7 | 3.0 |
| 5 | 10.5 | 83.2 | 6.3 | | | | | | |

The results show that the reactions with 60 and 90 mg enzyme performed more or less comparable indicating idle enzyme at higher enzyme amount (substrate limitation). There are slight differences with regards to the use of 30 mg/ml CalB: after 1 h, about 50 mol % PDMA are formed using 3 mg/ml CalB, whereas at the same time already about 80 mol % PDMA are formed using either 6 or 9 mg/ml CalB. These differences are more distinct at the beginning of the reaction, i.e. within the first 2 to 3 hours. The Selwyn plot (data not shown) showed no indication for enzyme deactivation during the course of the reaction. The initial activity of the reaction with 30 and 60 mg enzyme were also comparable (5.34 and 5.45 U/mg enzyme), indicating regular Michaelis-Menten kinetics. The results clearly indicate a higher hydrolysis rate of PDMA with higher enzyme amount.

Next, reactions at different pH were set-up using 19.6 wt % of PDDA in 10 ml reactions, 28° C., 6 mg/ml CalB, 8 ml KP, buffer 50 mM pH 7.5. The pH was set at 7.0, 7.5 or 8.0 using 5 M NaOH as titrant.

The reaction progress showed indications of enzyme deactivation, compared to the smooth reaction progress of the experiments testing different amounts of enzymes (see above). The observed potential enzyme inactivation is possibly due to the higher PDDA concentration. No significant differences were observed, in reaction progress at the different pH values (see Table 4).

TABLE 4
Conversion of PDDA to PDMA at different pH using CalB liquid.
Composition reaction mixture (mol %)

| Reaction | pH = 7 | | | pH = 7.5 | | | pH = 8 | | |
|---|---|---|---|---|---|---|---|---|---|
| time (h) | PD | PDMA | PDDA | PD | PDMA | PDDA | PD | PDMA | PDDA |
| 0 | 0 | 0 | 100 | 0 | 0 | 100 | 0 | 0 | 100 |
| 0.5 | 1.2 | 39.3 | 59.5 | 1.3 | 42.6 | 56.1 | 1.3 | 40.1 | 58.6 |
| 1.5 | 3.3 | 62.2 | 34.5 | 4.3 | 64.3 | 31.4 | 3.8 | 63.1 | 33.2 |
| 3 | 6.4 | 75.7 | 17.9 | | | | 7.5 | 76 | 16.5 |
| 3.5 | | | | 11 | 77.3 | 11.6 | | | |
| 4.5 | 9 | 81.9 | 9.1 | 14.2 | 78.5 | 7.4 | 11.2 | 80.3 | 8.6 |

In another set of experiments different titrants were tested for their impact on the PDDA conversion reaction. The reactions at 28° C. were set up as before, except that either 19.7 or 17.4 wt % PDDA was used in the presence of 16 mmol NaHCO₃ or using 5M NaOH as titrant.

The reaction progress of both reactions was comparable. No difference was observed. The productivity of the reaction with NaHCO₃ is 25% higher compared to reactions using 5 M NaOH. The pH of the reaction with NaHCO₃ starts around 8 and decreases during the reaction to about 7.4 (Table 5).

TABLE 5
Conversion of PDDA to PDMA using different titrants using CalB liquid.
Composition reaction mixture (mol %)

| Reaction | Titration with NaOH 5M | | | NaHCO₃ | | |
|---|---|---|---|---|---|---|
| time (h) | PD | PDMA | PDDA | PD | PDMA | PDDA |
| 0 | 0 | 0 | 100 | 0 | 0 | 100 |
| 0.5 | 1.3 | 42.6 | 56.1 | 1 | 40.6 | 58.4 |
| 1.5 | 4.3 | 64.3 | 31.4 | 3.8 | 65.2 | 31.1 |
| 3.5 | 11 | 77.3 | 11.6 | 11.5 | 79.2 | 9.3 |
| 4.5 | 14.2 | 78.5 | 7.4 | 15.2 | 79.2 | 5.6 |

A further experiment was performed testing the influence of higher temperature, i.e. shift from 28° C. and 6 mg/ml CalB to 37° C. and 7.5 mg/ml CalB together with NaOH as titrant. The performance was more or less the same, with some deviations in the PDDA conversion of the reaction at 37° C. which slowed down after 2-3 h, and that the reaction time was higher despite the higher enzyme amount. The hydrolysis rate of PDMA also slowed down at 37° C., indicating enzyme degradation at such high temperature (Table 6).

TABLE 6
Conversion of PDDA to PDMA at
different temperatures using CalB liquid.
Composition reaction mixture (mol %)

| Reaction | 28°/6 mg CalB/mL | | | 37°/7.5 mg CalB/mL | | |
|---|---|---|---|---|---|---|
| time (h) | PD | PDMA | PDDA | PD | PDMA | PDDA |
| 0 | 0 | 0 | 100 | 0 | 0 | 100 |
| 0.5 | 1.3 | 42.6 | 56.1 | 1.9 | 44.4 | 53.7 |
| 1 | | | | 3.3 | 56.3 | 40.4 |
| 1.5 | 4.3 | 64.3 | 31.4 | | | |
| 2 | | | | 5.9 | 68.4 | 25.7 |
| 3.5 | 11 | 77.3 | 11.6 | | | |
| 4 | | | | 9.8 | 77.6 | 12.6 |
| 4.5 | 14.2 | 78.5 | 7.4 | | | |
| 7 | | | | 14.3 | 78.7 | 7 |

In a parallel experimental approach, titration with NaHCO₃ was tested at 37° C. and either 3 mg/ml (37° C.) or 6 mg/ml (28° C.) CalB (17.4 wt % PDDA) and a pH of 7.2 to 7.5. There was no deactivation in the reactions at 37° C. (which was detected in the experiments using NaOH as titrant at 37° C.). The reaction proceeded quite fast using almost half the amount of enzyme. The hydrolysis rate of PDMA also increased at 37° C. The initial activity at 37° C. increased to 4.4 U/mg compared to 2.9 U/mg at 28° C. (Table 7).

TABLE 7
Conversion of PDDA to PDMA at different
temperatures with NaHCO₃ using CalB liquid.
Composition reaction mixture (mol %)

| | 3 mg CalB/mL; 37° C.; | | | 6 mg CalB/mL; 28° C.; | | |
|---|---|---|---|---|---|---|
| Reaction | NaHCO₃ | | | NaHCO₃ | | |
| time (h) | PD | PDMA | PDDA | PD | PDMA | PDDA |
| 0 | 0 | 0 | 100 | 0 | 0 | 100 |
| 0.5 | 1 | 37.5 | 61.5 | 1 | 40.6 | 58.4 |
| 1 | 2.6 | 54.5 | 42.9 | | | |
| 1.5 | | | | 3.8 | 65.2 | 31.1 |
| 2 | 6.6 | 70.9 | 22.5 | | | |
| 3.5 | | | | 11.5 | 79.2 | 9.3 |
| 4 | 15.6 | 77.6 | 6.8 | | | |
| 4.5 | | | | 15.2 | 79.2 | 5.6 |

Further testing was performed to compare the productivity using different amounts of PDDA together with NaHCO₃(Table 8). The results indicate some enzyme deactivation by increased PDDA concentration.

TABLE 8
Best results with CalB liquid and NaHCO₃.

PDDA [wt %]

| | 14.2 | 17.5 |
|---|---|---|
| Reaction time (h) | 5 | 6 |
| Composition mixture at 7 h (mol %) | | |
| PDMA | 83 | 79 |
| PD | 12 | 13 |
| PDDA | 5 | 8 |
| Concentration PDMA end reaction (wt %) | 9 | 10 |
| Productivity (g PDMA/L*h) | 19 | 19 |
| Enzyme usage (kg/kg PDMA) | 0.025 | 0.03 |

Summarizing our testing, we detected some enzyme deactivation during the reaction, which was favored by both increased PDDA concentration, i.e. PDDA concentrations >17 wt %, and/or at higher temperature (e.g. 37° C.). However, this deactivation could be partially suppressed in the presence of 10 wt % NaHCO₃. No increase in productivity was obtained by 20% increase in PDDA concentration, however, higher CalB concentrations let to increased hydrolysis of PDMA. The yield towards PDMA dropped under these conditions. Thus, in order to obtain best results regarding selectivity and productivity in the conversion of PDDA to PDMA using liquid form of CalB are conditions at 28° C., 14-15 wt % PDDA, 2 mg/ml CalB together with NaHCO₃.

### Example 4: Reaction Optimization for CalB (Immobilized Form)

Several commercially available CalB preparations have been tested including Novozym®435 (Novozymes), Immozyme-CalB-T2-150XL (Chiral Vision), Fermase (Fermenta) and CLEA immob (CLEA Technologies).

For a first test, different amounts of immobilized enzymes, based on the activity data given by the suppliers, were tested in 10 ml reactions (see Table 9) and the initial activity of the enzymes determined (Table 10).

TABLE 9
Reaction set up with titration and 24 wt % PDDA together
with 25 mg Immozyme-CalB-T2-150XL, 30 mg CLEA immob
or 50 mg Novozym ®435 or Fermase.

| Compound | Amount | | |
|---|---|---|---|
| PDDA (91%) [g] | 2.7 | | |
| KPᵢ buffer | 7.5 | | |
| 50 mM pH 7.5 [ml] | | | |
| pH | 7.5 | | |
| CalB immobilized [mg] | 25 | 30 | 50 |

| | |
|---|---|
| Temperature [° C.] | 28 |
| NaOH [M] | 5 |

TABLE 10
Initial activity of immobilized enzymes.
[*] = activity according to supplier.

| | | | Initial activity |
|---|---|---|---|
| | | Activity | [U/g] |
| Name | Form | [U/g]* | on PDDA |
| Novozym ®435 | Adsorbed | >5000 | 4800 |
| Immozyme-CalB-T2-150XL | Covalent bond | 15000 | 8800 |
| Fermase | Covalent bond | 10000 | 300 |
| CLEA immob | Adsorbed | | 5500 |

The best results were achieved with Immozyme-CalB-T2-150XL, which is in line with the activity data from the supplier. The activity of Fermase was relatively low. Also, the selectivity of this enzyme formulation was lower compared to the others (data not shown). Using 50 mg Novozym®435, formation of 70 mol % PDMA was achieved after 1 h, compared to Fermase (50 mg) or CLEA immob (30 mg) with only 40 mol % PDMA formed after 1 h. With 25 mg Immozym-CalB-T2-150XL 60 mol % PDMA was formed after 1 h. The maximum mol % PDMA formation of 80% was achieved for instance after 1.5 h with Novozym®435 (Table 11).

TABLE 11
Conversion of PDDA to PDMA with different immobilized enzymes.
Composition reaction mixture (mol %)

| Reaction | Novozym ®435 | | | Immozyme-CalB-T2-150XL | | | Fermase | | | CLEA immob | | |
|---|---|---|---|---|---|---|---|---|---|---|---|---|
| time (h) | PD | PDMA | PDDA | PD | PDMA | PDDA | PD | PDMA | PDDA | PD | PDMA | PDDA |
| 0 | 0 | 0 | 100 | 0 | 0 | 100 | 0 | 0 | 100 | 0 | 0 | 100 |
| 0.5 | 1.9 | 45.6 | 51.6 | 1.3 | 40.4 | 58.3 | 1.6 | 26.9 | 71.5 | 1.0 | 30.4 | 68.6 |
| 1 | 5.2 | 69.9 | 24.9 | 3.3 | 58.8 | 37.9 | 7.4 | 55.5 | 37 | | | |
| 1.5 | 9.7 | 80.6 | 9.7 | | | | | | | 4.2 | 56.4 | 39.5 |
| 2 | 14.2 | 81 | 4.8 | | | | | | | | | |
| 2.5 | | | | 10.3 | 79.1 | 10.6 | 14.2 | 68.0 | 17.8 | 8.6 | 72.1 | 19.3 |
| 3.5 | | | | 14.5 | 79.6 | 5.9 | 21 | 72.5 | 6.4 | 13.4 | 78.0 | 8.6 |
| 4.5 | | | | | | | | | | 17.3 | 77.5 | 5.2 |

To test the influence of titrant, Novozym®435 (50 mg), Immozym-CalB-T2-150XL (25 mg) and Fermase (50 mg) were tested with 16 mmol NaHCO₃ (final concentration) instead of NaOH in 10 ml reactions and 22 wt % PDDA at 28° C. With this set-up, only a yield of 70 mol % PDMA could be achieved after 2 h (Novozym®435), 3 h (Immozym-CalB-T2-150XL) or 3.5 h (Fermase), see Table 12.

TABLE 12
Conversion of PDDA to PDMA with different immobilized enzymes using NaHCO₃.
Composition reaction mixture (mol %)

| | Novozym ®435 | | | Immozyme-CalB-T2-150XL | | | Fermase | | |
|---|---|---|---|---|---|---|---|---|---|
| Reaction | 50 mg | | | 25 mg | | | 50 mg | | |
| time (h) | PD | PDMA | PDDA | PD | PDMA | PDDA | PD | PDMA | PDDA |
| 0 | 0 | 0 | 100 | 0 | 0 | 100 | 0 | 0 | 100 |
| 0.5 | 1.6 | 38.9 | 59.5 | 0.5 | 25.0 | 74.5 | 1.1 | 24.6 | 74.3 |
| 1 | 4.2 | 57.7 | 37.1 | 1.5 | 39.9 | 58.6 | | | |
| 1.5 | 7.7 | 66.9 | 25.3 | | | | 5.2 | 52.9 | 41.8 |
| 2 | 12.1 | 71.4 | 16.5 | | | | | | |
| 2.5 | 16.9 | 72.5 | 10.6 | 8.0 | 67.6 | 24.5 | 12.3 | 64.6 | 23.1 |
| 3 | 22.1 | 70.4 | 7.5 | | | | | | |
| 3.5 | | | | | | | 23 | 69.3 | 7.7 |
| 4.5 | | | | 16.5 | 72.2 | 11.3 | | | |
| 5.5 | | | | 25.5 | 69 | 5.4 | | | |

We further evaluated the best conditions with Novozym®435, i.e. different concentrations of PDDA, different amount of titrant NaHCO₃ and/or different amounts of CalB. The temperature was set to 28° C. at a pH of 7.5. When testing different amounts of enzyme, it turned out that the initial activity decreased significantly using 50 mg Novozym®435 indicating that under these conditions too much enzyme was present and a significant amount was idle. An increase in the hydrolysis rate of PDMA (towards formation of PD) could be detected with increasing enzyme and/or PDMA concentration. When testing different concentrations of PDDA, relatively easy conversion of PDDA at 14.1 wt % using 50 or 75 mg Novozym®435 could be shown. At higher concentration, the hydrolysis rate of PDMA increases leading to overall lower yield. The highest initial activity at lowest enzyme amounts (1 mg/mL) indicate that not all enzyme is busy at higher enzyme amount (1.5 mg/mL). The results are shown in Table 13 and 14.

TABLE 13
Conversion of PDDA to PDMA with different amounts Novozym ®435.
Composition reaction mixture (mol %)

| | Novozym ®435 | | | Novozym ®435 | | | Novozym ®435 | | |
|---|---|---|---|---|---|---|---|---|---|
| Reaction | 15 mg | | | 25 mg | | | 50 mg | | |
| time (h) | PD | PDMA | PDDA | PD | PDMA | PDDA | PD | PDMA | PDDA |
| 0 | 0 | 0 | 100 | 0 | 0 | 100 | 0 | 0 | 100 |
| 0.5 | 0.5 | 19.7 | 79.8 | 1.3 | 38.3 | 60.4 | 1.9 | 45.6 | 52.6 |
| 1 | | | | | | | 5.2 | 69.9 | 24.9 |
| 1.5 | | | | 6.9 | 70.1 | 23 | 9.7 | 80.6 | 9.7 |
| 2 | | | | | | | 14.2 | 81 | 4.8 |
| 2.5 | 3.9 | 55.1 | 41 | 13.6 | 79.3 | 8.1 | | | |
| 3 | | | | 16.7 | 77.9 | 5.3 | | | |
| 3.5 | | | | 20.3 | 75.9 | 3.8 | | | |
| 4 | 8.6 | 75 | 16.7 | | | | | | |
| 5.5 | 13.4 | 79.3 | 7.3 | | | | | | |
| 6 | 15.2 | 79.8 | 5.0 | | | | | | |

TABLE 14
Conversion of PDDA to PDMA with different amounts
Novozym ®435, different concentrations PDDA using NaHCO₃.
Composition reaction mixture (mol %)

| | 14.2 w % PDDA | | | 17.9 w % PDDA | | | 21.6 w % PDDA | | |
|---|---|---|---|---|---|---|---|---|---|
| Reaction | 15 mg Novozym ®435 | | | 50 mg Novozym ®435 | | | 50 mg Novozym ®435 | | |
| time (h) | PD | PDMA | PDDA | PD | PDMA | PDDA | PD | PDMA | PDDA |
| 0 | 0 | 0 | 100 | 0 | 0 | 100 | 0 | 0 | 100 |
| 0.5 | 0.8 | 28.9 | 70.4 | 1.5 | 37.8 | | 1.6 | 38.9 | |
| 1 | | | | | | | 4.2 | 57.7 | |
| 1.5 | 3 | 61.3 | 35.7 | 8.2 | 74.2 | | 7.7 | 66.9 | |
| 2 | | | | 14.8 | 78.5 | | 12.1 | 71.2 | |
| 2.5 | | | | | | | 16.9 | 72.5 | |
| 3 | 8.2 | 83.3 | 8.5 | 27.3 | 69.4 | | 22.1 | 70.4 | |
| 4 | 11.8 | 84.3 | 3.8 | | | | | | |
| 5 | 16.2 | 81 | 2.8 | | | | | | |
| 6 | 20.3 | 77.3 | 2.4 | | | | | | |

Similar evaluations had been made with Immozym-CalB-T2-150XL i.e. 10 ml reactions with different concentrations of PDDA (14.2 wt %, 14.1 wt %, 17.4 wt %), different amount of NaHCO₃ (1 g, 1 g, 1.3 g) and/or different amounts of CalB (15 mg, 30 mg). The temperature was set to 28° C. at a pH of 7.5 (Table 15). The performance was equivalent to what has been seen with Novozym®435. Again, PDMA yield decreases at higher concentrations because of increased hydrolysis of PDMA (Table 15).

TABLE 15
Conversion of PDDA to PDMA with different amounts
Immozym-CalB-T2-150XL, different concentrations PDDA using NaHCO₃.
Composition reaction mixture (mol %)

| | 14.2 w % PDDA | | | 14.1 w % PDDA | | | 17.4 w % PDDA | | |
|---|---|---|---|---|---|---|---|---|---|
| Reaction | 15 mg Immozyme-CalB-T2-150XL | | | 30 mg Immozyme-CalB-T2-150XL | | | 30 mg Immozyme-CalB-T2-150XL | | |
| time (h) | PD | PDMA | PDDA | PD | PDMA | PDDA | PD | PDMA | PDDA |
| 0 | 0 | 0 | 100 | 0 | 0 | 100 | 0 | 0 | 100 |
| 0.5 | 0.7 | 32.0 | 67.3 | 2.7 | 65.2 | 32.1 | 1.1 | 37.8 | 61.1 |
| 1 | 2.1 | 59.4 | 38.5 | 7.2 | 84.0 | 8.7 | 3.8 | 64.8 | 31.3 |
| 1.5 | 3.7 | 73.5 | 22.8 | 12.5 | 83.0 | 4.5 | 6.8 | 76.6 | 16.6 |
| 2 | 5.9 | 81.9 | 12.2 | 19 | 77.7 | 3.3 | 10.7 | 80.8 | 8.5 |
| 3 | 10.8 | 83.9 | 5.3 | | | | 18.7 | 76.9 | 4.4 |

### Example 5: Recycling Experiments Using Immobilized CalB

The recycle experiments were performed in custom made reactors (400 ml) in which the immobilized enzyme can be filtered off and left in the reactor without handling the enzyme. A glass frit was put just above the bottom tap to keep the biocatalyst in the reactor. To avoid blockage of the filter, the pore size of the frit was relatively small compared to the particle size of Novozym®435 and Immozym-CalB-T2-150XL (>300 μm). The stirrer type was chosen based on the frequently used stirrers in the productions vessel; RCI impeller. Two baffles were made on the glass wall (0.5 cm; internal diameter reactor=8 cm). The reactors (with jacket) were, in parallel, connected to a thermostat. The reaction temperature was checked with a Pt1000. The pH was checked regularly.

The recycle experiments were performed with 1 mg/ml enzyme (Novozym®435 or Immozym-CalB-T2-150XL) applying the following recipe (Table 16). At the end of the reaction the reaction mixture was filtered. After washing with phosphate buffer 50 mM pH=7.5 (15 mL), the enzyme was stored in the reactor in phosphate buffer 50 mM pH=7.5 (10 mL) at room temperature.

TABLE 16
Reaction set up in 250 ml with 14.2 wt % PDDA.

| Compound | Amount | |
|---|---|---|
| Demi water | 200 | ml |
| PDDA (91%) | 42 | g |
| Enzyme | 250 | mg |
| NaHCO₃ | 25 | g |
| Temperature | 28° | C. |
| Stirrer speed | 300 | rpm |
| TOTAL | 250 | ml |

With both enzymes, about 80 mol % PDMA was formed after about 4 hours, with reaching the maximum of about 85 mol % after 5 hours.

Using Novozym®435, 9 recycle experiments were performed over a period of 20 days. The reaction started as a three-phase system, PDDA does not completely dissolve, and accumulated as a clear liquid phase with Novozym®435 beads. The initial activity in the individual reactions gradually decreased (about 4%) with a parallel increase in reaction time to reach comparable conversions. After 9 recycle experiments, the reaction time increased from 6 h to 8 h. The remaining PDDA amount after 6 h increased from about 5 mol % to about 13 mol % (Table 17).

TABLE 17
Recycle experiments Novozym ®435.
Composition reaction mixture (mol %)

| | | | | Reaction 9 | | |
|---|---|---|---|---|---|---|
| Reaction | Reaction 1 | | | (after 20 days) | | |
| time (h) | PD | PDMA | PDDA | PD | PDMA | PDDA |
| 0 | 0 | 0 | 100 | 0 | 0 | 100 |
| 0.5 | 0.5 | 21.8 | 77.6 | | | |
| 1 | 1.2 | 39.5 | 59.3 | 0.7 | 28.7 | 70.6 |
| 2 | 3.4 | 62.0 | 34.6 | 1.5 | 47.6 | 50.9 |
| 3 | 5.5 | 73.3 | 21.2 | | | |
| 4 | 8.1 | 79.5 | 12.3 | 3.6 | 68.3 | 28.1 |
| 5 | 10.8 | 82.2 | 7.0 | | | |
| 6 | 14.0 | 81.6 | 4.4 | 6.4 | 80.6 | 13.0 |
| 7 | | | | 8.0 | 82.7 | 9.3 |
| 8 | | | | 9.9 | 84.6 | 5.6 |

To compensate for this loss in activity and keep the reaction time more or less the same extra enzyme could be added to the each of the recycle experiments (or every five reactions/recycle experiments). After 25 recycle experiments/reactions the total amount of enzyme in the reaction was doubled (from 1 kg/m³ to 2 kg/m³).

When using Immozym-CalB-T2-150XL under the conditions as described above, the results were more or less the same: the average decrease in initial activity was about 3% which is somewhat less compared to Novozym®435. The reaction time gradual increased from 6 h to 8 h. The remaining PDDA amount after 6 h increased from about 5 mol % to about 14 mol %. Also, these results are comparable to the Novozym®435 recycling results (Table 18). Thus, not much differences were observed between covalently bound immobilized enzyme (Immozym-CalB-T2-150XL) and non-covalently bound, adsorbed, immobilized enzyme (Novozym®435) in 9 recycling experiments.

TABLE 18
Recycle experiments Immozym-CalB-T2-150XL.
Composition reaction mixture (mol %)

| | | | | Reaction 9 | | |
|---|---|---|---|---|---|---|
| Reaction | Reaction 1 | | | (after 20 days) | | |
| time (h) | PD | PDMA | PDDA | PD | PDMA | PDDA |
| 0 | 0 | 0 | 100 | 0 | 0 | 100 |
| 0.5 | 0.5 | 24.6 | 74.9 | | | |
| 1 | 1.0 | 38.7 | 60.3 | 0.5 | 31.3 | 68.1 |
| 2 | 3.1 | 62.5 | 34.4 | 1.3 | 49.6 | 49.0 |
| 3 | 4.7 | 73.5 | 21.7 | | | |
| 4 | 6.8 | 80.1 | 13.1 | 3.5 | 70.5 | 26.0 |
| 5 | 9.0 | 82.7 | 8.3 | | | |
| 6 | 11.1 | 83.9 | 5.0 | 5.9 | 79.8 | 14.3 |
| 7 | | | | 7.2 | 82.8 | 10.0 |
| 8 | | | | 8.7 | 84.9 | 6.3 |

Our experiments revealed the best outcome regarding selectivity and productivity with conditions at 28° C., NaHCO₃, 14 to 15 wt % PDDA and 1 mg/ml immobilized enzyme. When recycling the enzyme 9× over a period of 20 days, the average activity loss was in the range of 3 to 4%.

### Example 6: Isolation of PDMA

After the reaction with either liquid or immobilized CalB, PDMA was isolated by extraction with dichloromethane (DCM) as solvent. Proteins (including the CalB) which might be present in the DCM layer have to be removed by (ultra)filtration prior to extraction of PDMA.

Best results were obtained in the presence of 10 wt % NaCl added after filtration but before DCM extraction leading to an increase in distribution coefficient for PDMA in DCM compared to extraction without NaCl addition (increase from 1.4 to about 3). With about 30 vol % DCM after 4 extractions, a yield of about 95% was obtained.

1. A process for production of 1,3-propanediol monoacetate (PDMA) comprising mono-deacetylation of 1,3-propanediol diacetate (PDDA) catalyzed by carboxylic ester hydrolases [EC 3.1.1], preferably esterases or lipases, more preferably lipases [EC3.1.1.3], in particular Candida lipase, preferably Candida antarctica lipase B (CalB).

2. Use of carboxylic ester hydrolases [EC 3.1.1], preferably esterases or lipases, more preferably lipases [EC3.1.1.3], for mono-deacetylation of PDDA into PDMA.

3. Process or use according to claim 1, wherein the lipase is used in liquid or immobilized form.

4. Process or use according to claim 3, wherein the immobilized enzyme is adsorbed or covalently bond.

5. Process or use according to claim 3, wherein the enzyme is re-used for at least 5 times.

6. Process or use according to claim 1, wherein PDDA is converted into PDMA at a rate of at least about 75%.

7. Process or use according to claim 1, wherein the mono-deacetylation reaction is in the presence of NaHCO₃.

8. Process or use according to claim 1, wherein the mono-denitrification reaction is conducted at pH in the range of 7 to 8.

9. Process or use according to claim 1, wherein the mono-deacetylation reaction is conducted at 28° C.

10. Process or use according to claim 1, wherein the amount of PDDA is less than 40 wt %.
